(19) United States
(12) Patent Application Publication
(10) Pub. No.: US 2011/0178267 A1
(43) Pub. Date: Jul. 21, 2011

(54) Novel organic materials and methods for obtaining thereof

(76) Inventor: Vladimir Tarnovsky, Fair Lawn, NJ (US)

(21) Appl. No.: 12/657,194

(22) Filed: Jan. 15, 2010

(51) Int. Cl. C08G 63/06; C07K 1/00; C07C 229/00; C07H 5/06; C07H 1/00; C07D 473/04

(52) U.S. Cl. 528/361; 530/402; 562/553; 536/55.3; 536/124; 544/274

(57) ABSTRACT

Methods for obtaining Tamaritt and Tamaritt-Intermediate based materials are implemented through a secondary chemical reactor providing a second chemical reaction conducted with at least two reactants. The first reactant is Tamaritt and the second is an inorganic, or organic, or bio-molecular reagent, or their combination. Tamaritt is produced in a primary chemical reactor, modulating chemical reactions, preferably using a plasma device, and producing certain byproducts. A solid organic dye is dissolved in water producing a solution placed into the primary reactor, wherein the temperature and pressure are predeterminedly adjusted. The byproducts are injected into the solution, thereby commencing a first chemical reaction conducted until it finally results in obtaining Tamaritt. Tamaritt-Intermediate byproducts, being bond-altering catalyst-reagents, are obtained at a certain stage of the process. The resulted Tamaritt-Intermediate byproducts can act as separate agents or, in combinations, can participate in novel reactions with organic solutions producing distinct and useful materials.

### CROSS-REFERENCE TO RELATED APPLICATIONS

The present patent application is closely related to a U.S. patent application Nr. 12/583,273 “Organic material possessing novel properties, method, and apparatus for making the same” filed on 18 Aug. 2009 by the instant inventor, which application is hereby incorporated by reference in its entirety.

### FIELD OF THE INVENTION

The present invention relates to the field of chemical technologies, particularly to the plasma-assisted and thermo-assisted chemical technology.

### BACKGROUND OF THE INVENTION

As well known, organic based materials are widely produced and consumed in a variety of forms, from plastics to semiconducting polymers, artificial enzymes, and biocompatible implants. This invention discloses new chemical materials with a wide range of useful properties, and methods for making these materials.

The aforementioned U.S. patent application Nr. 12/583,273 has introduced a new organic material (herein called ‘Tamaritt’) that possesses a number of highly functional properties, such as: inertness in all acids, bases, and other known solvents; ability to be affected by external electric fields in solutions; initiation of chemical reactions on its surface; stability in high temperature environments; active interaction with bio-molecular substances, etc. According to present knowledge of the instant inventor, Tamaritt can be defined as a plurality of families of functionalized organic-based polymers (‘Tamaritt families’, or ‘Tamaritts’), wherein each family corresponds to a particular organic dye (pH indicator/biological stain) that may act as solvents/catalysts, reactants/reagents, or any combination thereof, depending on their chemical environment.

According to an embodiment described in the U.S. patent application Nr. 12/583,273, Tamaritt is particularly produced through a method comprising the steps of:—providing a primary chemical reactor, in some embodiments, including a vessel associated with a plasma device capable of initiating a plasma discharge that produces predetermined byproducts;—dissolving a solid dye in water, obtaining a predetermined dye solution that is placed in the vessel;—elevating a temperature and adjusting a pressure inside the vessel up to predetermined temperature and pressure respectively;—injecting the byproducts into the dye solution thereby commencing a chemical reaction, the reaction proceeds during a predetermined time, and further results in obtaining Tamaritt; and—extracting Tamaritt from the dye solution.

Further, according to the U.S. patent application Nr. 12/583,273, other chemical reactor embodiments comprise means for heating the solution for modulating properties of Tamaritt. Several apparatus embodiments have also been described in the U.S. patent application Nr. 12/583,273, including those with an additional chemical reactor joined with the primary chemical reactor further modifying the obtained Tamaritt.

Thusly, according to the U.S. patent application Nr. 12/583,273, “The inventive organic material can be produced by: (A) an apparatus including a plasma device with no external heating means; or (B) an apparatus including external heating means with no plasma device; or (C) an apparatus including external heating means and a plasma device, which options A, B, and C represent the plasma device and/or the heating means as modulators of a chemical reaction necessary for production of the inventive organic material.” Hence, the present invention contemplates the use of members of the Tamaritt families, produced by apparatus means of any of the options: A (‘plasma-Tamaritt’ apparatus), or B (‘thermo-Tamaritt’ apparatus), or C (‘plasma-thermo-Tamaritt’ apparatus). On the other hand, the plasma device and/or the external heating means are means for modulating chemical reactions, which reactions are capable of producing Tamaritt and Tamaritt related products.

Since the dye ingredient in the foregoing method can be chosen of various types of organic dyes, it would be correct to define Tamaritt as a plurality of families of organic products that might be produced through the aforesaid methods using the A, or B, or C apparatus options, which methods generally refer to continuous condensation of an initial dye solution in water (liquid reactants) into a solid phase product, whose underlying chemical structure is independent on the A, B, and C options. These results indicate that in broad range conditions (T temperature, P pressure, R radical initiators) in the process of chemical reaction/rearrangement of certain dyes, the structure of the final solid condensate is encoded in the chemical structure of the initial dye.

### OBJECTS AND BRIEF SUMMARY OF THE INVENTION

The instant inventor recently discovered new facts and features related to Tamaritt, which further broaden the scope of chemical materials that might be obtained with the use of Tamaritt and its intermediate byproducts (herein called ‘Tamaritt-Intermediate byproducts’) generated in a primary chemical reactor at certain stages of the Tamaritt making processes, described in to the U.S. patent application Nr. 12/583,273. The certain stages are defined within a time period less than a predetermined time period for obtaining Tamaritt in a particular zone of the primary chemical reactor. For a primary reactor comprising a plasma device, it was established that the process taking place in the plasma device produces certain plasma reactive byproducts. During further chemical reactions of the plasma reactive byproducts with initial liquid reagents, substances are generated, which substances may be regarded as plasma-assisted Tamaritt-Intermediate byproducts, being bond-altering catalyst-reagents.

Likewise, for a primary reactor comprising an external heating means, during the external heating of initial liquid reagents and subjecting them to predetermined pressure conditions, as described in to the U.S. patent application Nr. 12/583,273, certain reactive byproducts are produced. The byproducts further react with initial liquid reagents, generating substances, which substances may be regarded as thermo-assisted Tamaritt-Intermediate byproducts, being bond-altering catalyst-reagents. Similar to the plasma-assisted Tamaritt-Intermediate byproducts, the thermo-assisted Tamaritt-Intermediate byproducts are obtained at certain stages of the Tamaritt making processes, described in to the U.S. patent application Nr. 12/583,273, and the stages are defined within a time period less than a predetermined time period for obtaining Tamaritt in a particular zone of the primary reactor.

The bond-altering catalyst-reagents (Tamaritts or Tamaritt-Intermediate byproducts) each can act as a separate agent, or in combination with the others can participate in a number of novel chemical reactions with organic solutions and, in particular, with organic dyes that allows producing a new class of distinct and useful materials.

Therefore, a first object of the present invention is to provide a new class of materials possessing useful properties, herein further called Tamaritt based materials', produced through specific chemical reactions conducted with certain reactants, wherein one of the reactants is a member of a Tamaritt family referred to a specific organic dye, which member is produced at the end of the Tamaritt making process using the aforementioned apparatus options A, or B, or C, described in the U.S. patent application Nr. 12/583,273. The first object is achieved by disclosing herein examples of such specific chemical reactions.

A second object of the present invention is to provide a class of materials, herein further called ‘Tamaritt-Intermediate based materials’, which materials are produced through specific chemical reactions conducted with at least two reactants, wherein at least one of the reactants is a Tamaritt-Intermediate byproduct (i.e. factually a Tamaritt-Intermediate dye) obtained at a certain stage of the Tamaritt making process using the aforementioned means for modulating the specific chemical reactions. The certain stage is defined within a time period less than a predetermined time period for obtaining Tamaritt in a particular zone of the primary reactor. The means for modulating the specific chemical reactions are implemented based on the apparatus options A, or B, or C, described in the U.S. patent application Nr. 12/583,273, i.e. factually being: plasma-assisting, thermo-assisting, and plasma-thermo-assisting means.

According to the instant invention, during the specific chemical reactions, a number of Tamaritt-Intermediate based materials are produces that have novel and useful properties. The Tamaritt-Intermediate based materials can be classified as organic dyes with the pH range between 2.5 and 7.5, whose color range may extend from green to violet. While being novel dyes, they are very powerful radical initiators as well.

According to the instant invention, the aforesaid Tamaritt-Intermediate byproducts can be extracted from the primary chemical reactor in two ways: (a) essentially with Tamaritt, and (b) essentially without Tamaritt, depending on a particular reactor zone of extraction. The extracted Tamaritt-Intermediate byproducts may vary depending on the time period from the beginning of the Tamaritt making process to the time of extraction. The extraction time for the Tamaritt-Intermediate byproducts is generally less than the predetermined time period for obtaining Tamaritt in the primary reactor. The extracted Tamaritt-Intermediate byproducts with or without Tamaritt can further be employed as reactants respectively in a secondary reactor (which, in some embodiments, may be optionally combined with a plasma device as well) in various chemical reactions producing novel materials (i.e. Tamaritt-Intermediate based materials) with useful properties. The second object is achieved by disclosing herein examples of such chemical reactions in the secondary reactor.

A third object of the present invention is to provide modifications to the already described Tamaritt making methods, which modifications represent multistage fractional extraction/dilution processes verses a continuous condensation process of liquid reactants into a solid phase product mentioned above.

According to the instant invention, in addition to the known steps of Tamaritt making methods, described in the U.S. patent application Nr. 12/583,273, an additional step is provided that comprises a repeated removal of a predetermined fraction of the contents (substantially in the form of solution, containing an amount of Tamaritt-Intermediate byproducts) from the primary reactor, and inputting a replacement amount of water preferably equal to the removed predetermined fraction. Such cyclic removal/dilution step can be repeated preferably until the removed fraction would contain only water of the same type as the added one. All Tamaritt-Intermediate byproducts extracted according to this method can be characterized at least by: its color, pH, mixing capacity with organic solvents, and capability of initiating radical polymerization.

Other objects of the present invention can become apparent to one skilled in the art upon learning the present disclosure.

### BRIEF DESCRIPTION OF THE DRAWINGS

FIG. 1—Prior Art illustrates a general scheme of an apparatus assembly for production of Tamaritt, according to an embodiment, described in the U.S. patent application Nr. 12/583,273.

FIGS. 2 and 2A (for a cyclic process) illustrate block-diagrams for production of the inventive Tamaritt based materials according to embodiments of the present invention.

FIGS. 3 and 3A (wherein the secondary reactor includes a plasma device) illustrate block-diagrams for production of the inventive Tamaritt-Intermediate based materials according to other embodiments of the present invention.

FIGS. 4 and 4A (wherein the secondary reactor includes a plasma device) illustrate block-diagrams for production of the inventive Tamaritt-Intermediate based materials according to other embodiments of the present invention.

FIG. 5 illustrates a block-diagram for production of the inventive Tamaritt-Intermediate byproducts and Tamaritt-Intermediate materials based on the Tamaritt-Intermediate byproducts, according to another embodiment of the present invention.

Identical reference numerals on the drawings generally refer to the same elements, unless otherwise is stated in the description. A newly introduced numeral in the description is enclosed into parentheses.

### PREFERRED EMBODIMENTS OF THE PRESENT INVENTION

While the invention may be susceptible to embodiment in different forms, there are shown in the drawings, and will be described in detail herein, specific embodiments of the present invention, with the understanding that the present disclosure is to be considered an exemplification of the principles of the invention, and is not intended to limit the invention to that as illustrated and described herein.

### Equipment

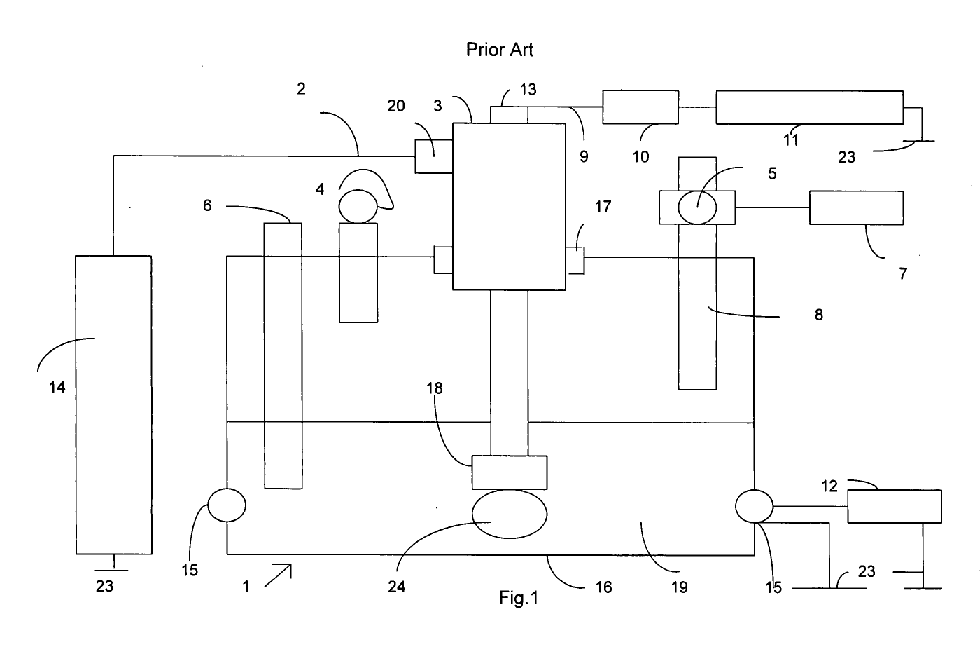

According to a preferred embodiment described in the U.S. patent application Nr. 12/583,273, Tamaritt is a new composite organic-based solid material that can be produced from an organic dye, particularly by combination of the external thermal heating (e.g. electro-heating, and/or microwave heating, though other known types of external heating can be suitably utilized as well) and adjusted pressure, and/or plasma-induced non-equilibrium rearrangements of the dye in a primary chemical reactor (1), whose preferred embodiment is depicted on FIG. 1—Prior Art.

As shown on FIG. 1—Prior Art, the chemical reactor 1 (see option C above) is essentially fed from a gas cylinder (14). The reactor 1 comprises a plasma device (3), having an inlet communicating with the gas cylinder 14 via a gas supply line (2) and a flow meter (20). The plasma device 3 is furnished with a high voltage connector (13) and a sealed screw connector (17).

The reactor 1 comprises a reaction vessel (16) wherein a dye solution (19) is supplied into via a junction connector (26). The dye solution can be chosen in a predetermined concentration, including the case where the dye is used in the dry form, i.e. without any solution in water.

The vessel 16 is covered by a removable lid. The vessel 16 is assembled with the plasma device 3 through the sealed connector 17. The interior of vessel 16 communicates with the plasma device 3 via an exit (outer) cup (18). A plasma reaction volume is operatively formed in a zone (24) proximate to the cup 18, which reaction produces predetermined byproducts. The vessel 16 also comprises a pressure gauge (4), a temperature control device (6), and a vent valve assembly (8), including a pressure vent valve (5) preferably communicating with a condenser (7), resupplying gas into the gas cylinder 14.

As shown on FIG. 1—Prior Art, a high-voltage power source (11) supplies a predetermined DC voltage via a load resistor box (10) used for electric current control, and via a high-voltage power cable (9), electrically connected with the box 10 to the plasma device 3. The electric current substantially causes producing plasma in the plasma device 3.

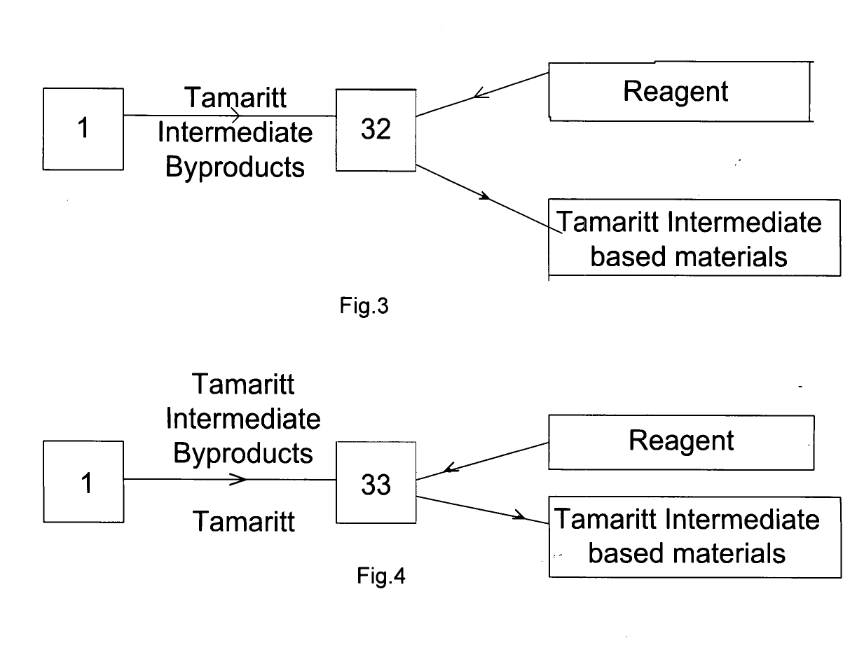

The reactor 1 comprises a number of thermal heating devices represented, for example, by electro-heating coils (15) acting as chemical reaction modulators. The coils 15 are connected with an electric power supply source (12) connected to a grounding (23). In particular embodiments, the heating devices, at least in part, can be represented by microwave heating devices that could be arranged, for example, around the vessel 16, as shown on FIG. 3. The coils 15 and the power source 12 produce an additional degree of control of the pressure and temperature of the interior of the vessel 16. The coils 15 and/or a direct plasma injection mechanism (described in the U.S. patent application Ser. No. 12/201,229 by the instant inventor, hereby entirely incorporated by reference), or any combination thereof can essentially initiate the chemical reaction in the reactor 1. An example of operation of the above-described equipment disclosed in the U.S. patent application Ser. No. 12/583,273 follows below.

Voltage and electric current supplied to the plasma device 3 initiate a plasma discharge through the feeding gas, so the byproducts of the discharge are directly injected into the chemical reaction vessel 16.

The gas outlet allows the gas and vapor to exit the reaction vessel 16 via the pressure valve 5 that can control the flow rate of the escaping gases and vapor.

Exemplarily, a solid organic dry dye is dissolved in water (preferably, distilled water) and the dye solution 19 is obtained. The dye solution is prepared in any desirable concentration and placed inside the chemical reaction vessel 16. In a special case, the dye is processed in the reactor in the dry form without introducing any water, as mentioned above.

The pressure inside the vessel 16 is adjusted in a range of 1-5 atmosphere or higher, (preferably 1.5 atmosphere), and the temperature inside the vessel 16 is adjusted to a value chosen from the range of from 20 to 200.degree.C, using the heating coils 15 (or possibly the heat from the plasma device 3) and the pressure valve 5.

A direct injection of the plasma reactive materials (byproducts) is then initiated into the dye solution 19 in the interior of vessel 16. The dye solution 19 is contacted with the plasma byproducts commencing the chemical reaction. The rate of reaction can be modulated by adjusting the electric current through the coils 15, the plasma conditions, or the flow rate. The reaction proceeds for a predetermined time, preferably several hours (preferably in the range from 1 to 8 hours) per 100 mL of solution. The timing of reaction in the reactor 1 is generally determined by any lack of color of the dye solution 19 and visible precipitation of the resultant solid material in the solution, which material is then extracted from the solution.

### Reactions Producing Tamaritt Based Materials—First Embodiment

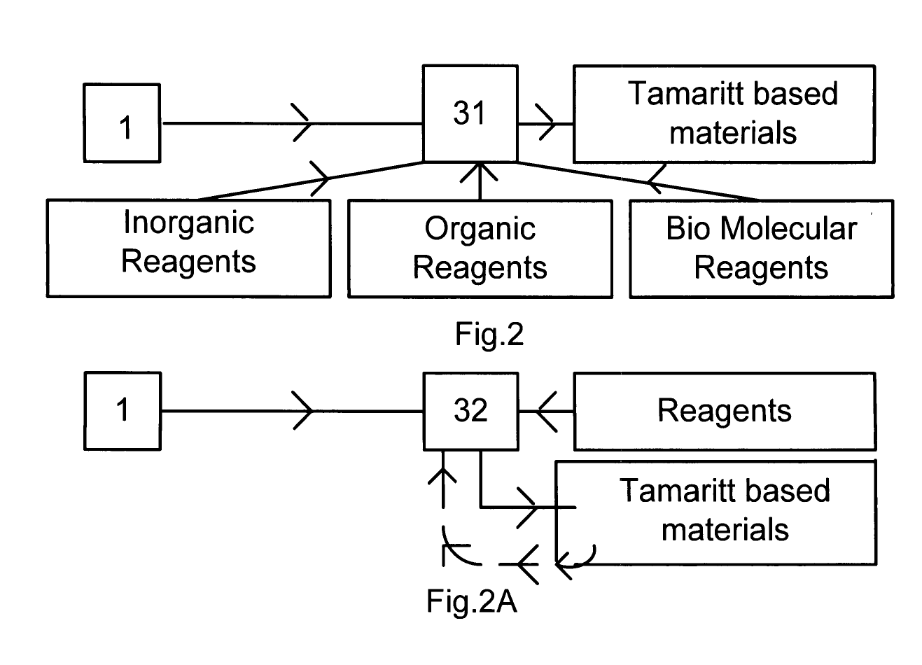

According to a First Embodiment the present invention, at least a portion of the contents of the primary chemical reactor 1 can be extracted and placed into a conventional chemical reactor (31), herein called a ‘secondary reactor’, particularly shown on FIG. 2. As illustrated on FIG. 2, a predetermined amount of Tamaritt is transferred from the primary reactor 1 to the secondary reactor 31, wherein one of the following materials can be added: an inorganic reagent (of course, including metals), or an organic reagent, or a bio-molecular reagent, or any combination thereof. In these cases, a respective chemical reaction takes place in the reactor 31, and a corresponding Tamaritt based material will be produced therein. The following examples demonstrate several types of the First Embodiment reactions.

Type (i). Tamaritt+Inorganic Reagents->New Tamaritt Based Materials

### Example 1

### Tamaritt+Inorganic Solvents->Fully Inert Tamaritt Based Material

### Example 2

### Tamaritt+Metal Nitrides->Tamaritt Based Material Incorporating Metal Cations and Negative Anions

### Example 3

### Tamaritt+Hydrogen Peroxide/Acetanilide>Dissolving Peroxide into Oxygen, Water+Tamaritt+Solution Derivative from Tamaritt (Radical\Initiator)

### Example 4

### Tamaritt+Precious Metal+Hydrogen Peroxide/Acetanilide->Metal Oxide (Metallic Colloid)+Tamaritt (Metal Condensate)

It is in particular established that the Tamaritt/Inorganic reactions of Example 4 may produce at least three new functionalized Tamaritt type materials: Metal/Tamaritt/Nitrites, Gold/Tamaritt, and Colloidal Gold extracted from gold. These reactions lead to production of neutral organic radicals by reacting Tamaritt with Hydrogen Peroxide/Acetanilide. The so produced organic radicals attack gold with an efficiency and speed exceeding those for any known method for chemically dissolving gold.

Type (ii). Tamaritt+Organic Reagents->New Tamaritt Based Materials

The Tamaritt/Organic reactions of such type are conducted with an organic reactant represented by a monomer that is capable to self-propagate once it starts reacting with Tamaritt. For instance, Tamaritt reacts with HEMA monomer, producing poly-(HEMA). More examples follow below.

### Example 5

### Tamaritt+Monomer(s)->Poly (Monomers)

### Example 5A

### Tamaritt+(2-Hydroxyethyl Methacrylate) HEMA->Poly (HEMA)

### Example 6

### Tamaritt+Hydrogen Peroxide/Acetanilide+(Monomers)->(Poly-monomers) Polymer;

### Example 7

### Poly-monomers (e.g. from Example 6)+Monomers->Poly-Monomers (Living Polymers)

This example demonstrates a successive (e.g. second) cycle (illustrated on FIG. 2a with a dashed line) of using a Tamaritt based material, obtained in a first cycle, in a reaction of the type (ii) with known organic reagents, in this case monomers. A number of such successive cycles can be empirically established based on the necessity in obtaining a Tamaritt based material with particular properties.

### Example 8

### Tamaritt+Hydrogen Peroxide/Acetanilide+Additives (Table Sugar)->Biopolymers

Type (iii). Tamaritt+Bio Molecular Material->New Tamaritt Based Materials

It's been established that Tamaritt and Tamaritt-Intermediate byproducts act as selective initiators/markers of the amino acid rearrangement and combinations, which results in a specific case of changing the solution's color as a function of the amino acid type and also as a function of time. In one example a combination of six amino acids in the presence of table sugar and 3% hydrogen peroxide/Acetanilide changed the color of the solution five times during five hours, from light green, blue, green, yellow, golden yellow. Reactions of this type indicate that Tamaritts might act similar to the DNA translation for making protein from amino acids in a cell. Some specific examples of reactions of this type follow below.

### Example 9

### Tamaritt+Complex Proteins->Protein Denaturing (Full Protein Degradation)

### Example 10

### Tamaritt/H2O2/Acetanilide+Amino Acids->Amino Acid Modification (Rearrangement)

### Example 11

### Tamaritt/H2O2/Acetanilide.+Amino Acids+Sugar (non reducing)->Amino-Sugar-Complex

### Example 12

### Tamaritt/H2O2/Acetanilide+Amino Acid+Table Sugar->Color Marking Product

### Example 13

### Tamaritt/H2O2/Acetanilide+Table Sugar->Poly-Sugars

### Reactions Producing Tamaritt-Intermediate Based Materials—Second and Third Embodiments

As noted above, Tamaritt-Intermediate based materials are produced through specific chemical reactions, wherein at least one of the reactants is a Tamaritt-Intermediate byproduct (i.e. factually a Tamaritt-Intermediate dye) obtained at a certain stage of the Tamaritt making process described in the U.S. patent application Nr. 12/583,273. According to the instant invention, the aforesaid Tamaritt-Intermediate byproducts can be extracted from the primary chemical reactor 1 in two ways: (a) essentially with Tamaritt (mostly in a predetermined lower zone of reactor) and (b) essentially without Tamaritt (mostly in a predetermined upper zone of reactor). The actual form of the Tamaritt-Intermediate byproducts depends on the time of extraction thereof from the primary reactor.

As depicted on FIG. 3, the extracted Tamaritt-Intermediate byproducts (essentially without Tamaritt) can further be employed respectively in a secondary reactor (32) in various chemical reactions producing Tamaritt-Intermediate based materials. FIG. 3A illustrates a secondary reactor 32 combined with a plasma device (34), similar to the plasma device 3. Therefore, according to a Second Embodiment of the present invention, at least a portion of the contents of the primary chemical reactor 1 can be extracted (in some cases under pressure) therefrom and moved into the secondary reactor 32. As illustrated on FIG. 3, predetermined amounts of Tamaritt-Intermediate byproducts are transferred from the primary reactor 1 to the secondary reactor 32, wherein one of the following materials can be added: an inorganic reagent, or an organic reagent, or a bio-molecular reagent (such as amino acids) or any combination thereof. In these cases, a respective chemical reaction takes place in the reactor 32, and a corresponding Tamaritt-Intermediate based material will be produced therein. More types of Tamaritt-Intermediate based materials can be produced with the use of the plasma device 34 for modifying the second chemical reaction.

### Example 14

### Tamaritt-Intermediate Byproducts+Solid Polymers=New Polymers

### Example 15

### Tamaritt-Intermediate Byproducts+Amino Acids->Amino Acids Rearrangement/Polymerization Accompanied by Change of Color of the Tamaritt-Intermediate Byproduct Solutions as a Function of the Amino Acids and Time

### Example 16

### Tamaritt-Intermediate Byproducts+HEMA (2-Hydroxyethyl Methacrylate)+Hydrogen Peroxide/Acetanilide->Poly-HEMA Resin

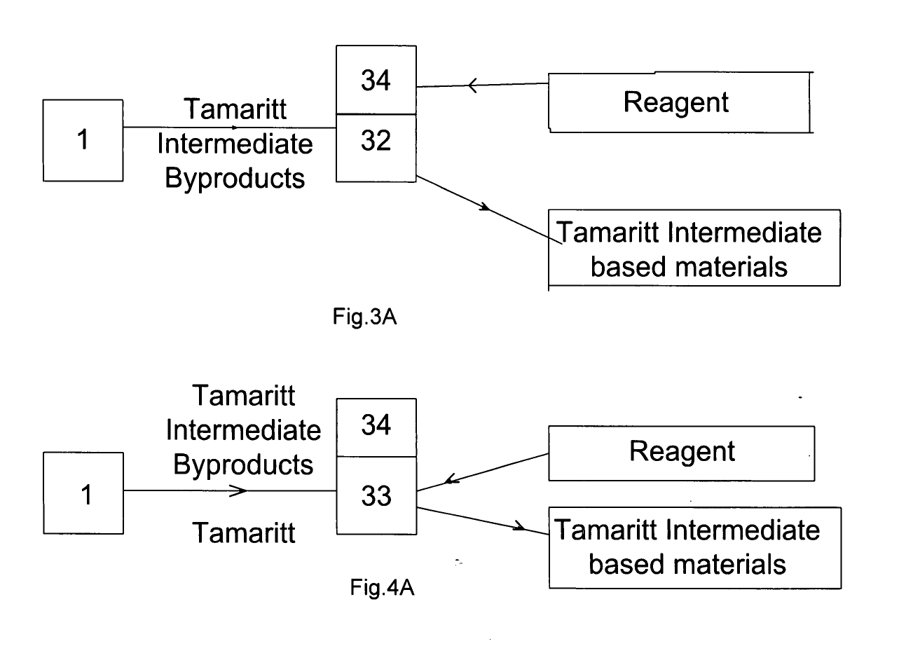

According to a Third Embodiment of the present invention, at least a portion of the contents of the primary chemical reactor 1 can be extracted and placed into a secondary reactor (33). As illustrated on FIG. 4, predetermined amounts of Tamaritt-Intermediate byproducts and a predetermined amount of Tamaritt are transferred from the primary reactor 1 to the secondary reactor 33, wherein one of the following materials can be added: an inorganic reagent, or an organic reagent, or a bio-molecular reagent. In these cases, a respective chemical reaction takes place in the reactor 33, and a corresponding Tamaritt-Intermediate based material will be produced therein. Examples of such reaction follow below.

### Example 17

### Tamaritt+Tamaritt-Intermediate Byproducts+Caffeine->Caffeine-Based Blue Dye

### Example 18

### Tamaritt+Tamaritt-Intermediate Byproducts+A Combination of Amino Acids->Poly-Amino-Tamaritt

### Gradual Removal and Dilution of Tamaritt Intermediates—Fourth Embodiment

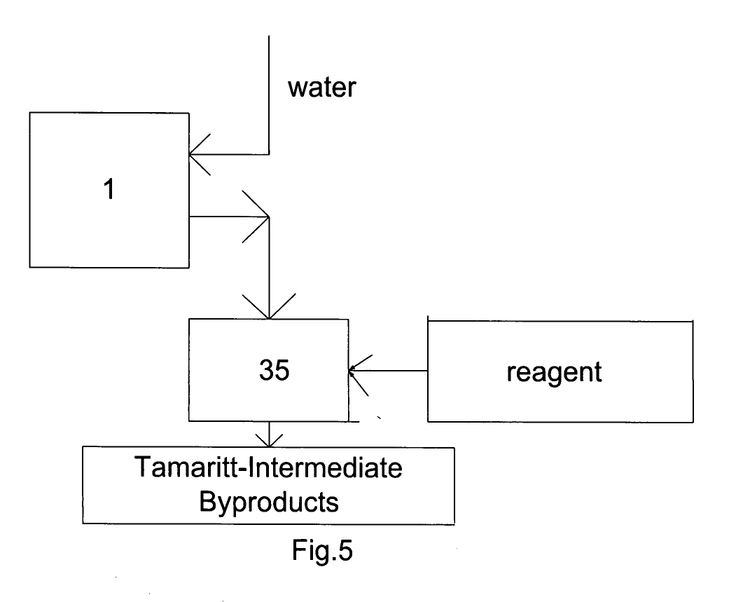

According to a Fourth Embodiment of the present invention, in addition to the known steps of Tamaritt making methods, described in the U.S. patent application Nr. 12/583,273, an additional step is provided that comprises a repeated removal of a predetermined fraction of the contents (substantially in the form of solution, containing an amount of Tamaritt-Intermediate byproducts) from the primary reactor 1, as depicted on FIG. 5, and inputting a replacement amount of water preferably equal to the removed predetermined fraction into the reactor 1. The removed fractions of Tamaritt-Intermediate byproducts can further be placed into a secondary reactor (35), shown on FIG. 5, to be employed for producing Tamaritt-Intermediate based materials. Similarly to the secondary reactors 31, 32, and 33, described above, the secondary reactor 35 can be combined with a plasma device (not illustrated). The removal-dilution step is repeated until the removed fraction would contain only water of the same type as the added one. All the Tamaritt-Intermediate byproducts extracted according to this method can be characterized at least by its color, pH, and capability of initiating radical polymerization.

In a particular embodiment, the extracted Tamaritt-Intermediate byproducts may further be placed in the secondary reactor 35, wherein they can be dissolved in aniline to separate the Tamaritt-Intermediate byproducts from water. The so purified Tamaritt-Intermediate byproducts cross-link with aniline, and remain in a gel-like state surrounded by the mixture of remaining aniline and water. The cross-link gel would encapsulate any anions from water, (e.g. Iodine ions), or phenols that are carrying negative OH functional groups. The following example demonstrates a chemical reaction of the Fourth Embodiment.

Tamaritt-Intermediate byproducts (4e) (fourth extract)+Aniline+Water->Tamaritt-Intermediate byproducts (4e)*Aniline (Tamaritt-Intermediate byproducts cross link with aniline in a gel-like liquid drops totally separate from the rest of solution)+Water.

I claim:

1. A method for obtaining Tamaritt based materials comprising the steps of:
providing a secondary chemical reactor;
providing a second chemical reaction in the secondary chemical reactor, said second chemical reaction results in obtaining said Tamaritt based materials, said second chemical reaction is conducted with at least two reactants, wherein a first reactant of said at least two reactants is a member of a Tamaritt family produced through a preceded method comprising the steps of:
a) providing a primary chemical reactor equipped with means for modulating chemical reactions, said means for modulating chemical reactions are capable of producing predetermined byproducts;
b) dissolving a solid organic dye in water obtaining a predetermined dye solution, said solution is placed into said primary reactor;
c) adjusting a temperature and a pressure inside said primary reactor up to a predetermined temperature and a predetermined pressure respectively;
d) injecting said byproducts into said dye solution thereby commencing a first chemical reaction; and
e) conducting said first reaction during a predetermined time period, until the first reaction finally results in obtaining at least said member of a Tamaritt family.

2. The method according to claim 1, wherein said at least two reactants include a second reactant being one of the following: an inorganic reagent, or an organic reagent, or a bio-molecular reagent, or any combination thereof.

3. The method according to claim 2, wherein said second reactant is an inorganic reagent being either an inorganic solvent, or metal, or metal nitrides.

4. The method according to claim 3, wherein said at least two reactants additionally include a third reactant being hydrogen peroxide/acetanilide.

5. The method according to claim 3, wherein said inorganic reagent is metal being gold.

6. The method according to claim 2, wherein said second reactant is an organic reagent being monomer.

7. The method according to claim 6, wherein said monomer is HEMA (2-Hydroxyethyl Methacrylate).

8. The method according to claim 6, wherein said at least two reactants additionally include a third reactant being hydrogen peroxide/acetanilide.

9. The method according to claim 2, wherein said second reactant is a bio-molecular reagent.

10. The method according to claim 9, wherein said bio-molecular reagent is either a complex protein, or an amino acid, or sugar.

11. The method according to claim 2, wherein said Tamaritt based material, obtained as a result of said method of claim 2, is further used as the first reactant in a predetermined number of successive cycles of said second chemical reaction.

12. The method according to claim 1, wherein said secondary reactor is combined with a plasma device.

13. A method for obtaining Tamaritt-Intermediate based materials comprising the steps of:
providing a secondary chemical reactor;
providing a second chemical reaction in the secondary chemical reactor, said second chemical reaction results in obtaining said Tamaritt-Intermediate based materials, said second chemical reaction is conducted with at least two reactants, wherein a first reactant of said at least two reactants is at least one Tamaritt-Intermediate byproduct produced through a preceded method comprising the steps of:
a) providing a primary chemical reactor, equipped with means for modulating chemical reactions, said means for modulating chemical reactions are capable of producing predetermined reactive byproducts in said primary reactor;
b) dissolving a solid organic dye in water obtaining a predetermined dye solution, said solution is placed into said primary reactor;
c) adjusting a temperature and a pressure inside said primary reactor up to a predetermined temperature and a predetermined pressure respectively;
d) injecting said reactive byproducts into said dye solution thereby commencing a first chemical reaction in said primary reactor, wherein said first reaction is capable of:—proceeding within a predetermined time period in a predetermined zone of said primary reactor,—producing at least one Tamaritt-Intermediate byproduct at a predetermined intermediate stage occurring before the end of said predetermined time period,—resulting in obtaining a member of a Tamaritt family at the end of said predetermined time period; and
e) extracting an amount of solution containing at least a fraction of said at least one Tamaritt-Intermediate byproduct from said predetermined zone of said primary reactor, and placing the extracted amount of solution into said secondary reactor.

14. The method according to claim 13, wherein a second reactant of said at least two reactants is selected from the group consisting of: a solid polymer, an amino acid, sugar, hydrogen peroxide/acetanilide, aniline.

15. The method according to claim 13, wherein a second reactant of said at least two reactants is HEMA, and a third reactant of said at least two reactants is Hydrogen Peroxide/Acetanilide.

16. The method according to claim 13, wherein the step (e) is modified as follows: extracting said at least one Tamaritt-Intermediate byproduct from said predetermined zone of said primary reactor, extracting said member of a Tamaritt family from another predetermined zone of said primary reactor, and placing the extracted at least one Tamaritt-Intermediate byproduct and the extracted member of a Tamaritt family into said secondary reactor, thereby providing said second chemical reaction.

17. The method according to claim 16, wherein a second reactant of said at least two reactants is caffeine, or a combination of amino acids.

18. The method according to claim 13, wherein
said preceded method comprising the additional steps of:
f) adding a predetermined weight amount of water into said primary reactor;
g) repeating the steps (c)-(f) a predetermined number of times.

19. The method according to claim 18, wherein the extracted amount of solution, containing at least a fraction of said at least one Tamaritt-Intermediate byproduct, is equal to said predetermined amount of water, and the step (g) is provided until the extracted solution contains only water of the same type as the added water.

20. The method according to claim 13, wherein said secondary reactor is combined with a plasma device.
